(19) United States
(12) Patent Application Publication
(10) Pub. No.: US 2026/0060514 A1
(43) Pub. Date: Mar. 5, 2026

(54) CAMERA CALIBRATION FOR SURGICAL SYSTEMS

(71) Applicant: Intuitive Surgical Operations, Inc., Sunnyvale, CA (US)

(72) Inventors: Wenqing SUN, Mountain View, CA (US); Maximilian H. ALLAN, Mill Valley, CA (US); Thomas KURMANN, Berne (CH); Albert HEBERLE, San Francisco, CA (US); Raymond YU, San Jose, CA (US)

(21) Appl. No.: 19/309,033

(22) Filed: Aug. 25, 2025

Related U.S. Application Data
(60) Provisional application No. 63/687,825, filed on Aug. 28, 2024.

(51) Int. Cl. A61B 1/00; A61B 1/06; G06T 3/18; G06T 7/00; G06T 7/80

(52) U.S. Cl. CPC A61B 1/00057; A61B 1/000095; A61B 1/0655; G06T 3/18; G06T 7/0014; G06T 7/85; G06T 2207/10068; G06T 2207/30168; G06T 2207/30204

(57) ABSTRACT

The present disclosure describes a system and method for camera calibration. The system includes a memory and a controller communicatively coupled to the memory. The controller moves an endoscope through a cannula and stops the endoscope in the cannula. The controller also captures, using the endoscope at the position, images of a reference corresponding to the cannula and adjusts a parameter of the endoscope based on the reference in the images.

### RELATED APPLICATION

This application claims the benefit of U.S. provisional patent application Ser. No. 63/687,825, filed Aug. 28, 2024, which is hereby incorporated herein by reference.

### TECHNICAL FIELD

The present disclosure relates generally to medical systems (e.g., digital fiducial systems, anatomy detection systems, clinical guidance systems, and surgical systems). Specifically, the present disclosure relates to a medical system that calibrates stereo cameras of an endoscope.

### BACKGROUND

Doctors use computer assisted medical systems to perform different medical tasks. For example, doctors may use computer assisted surgical systems to perform operations on patients, even remotely. These surgical systems use endoscopes with stereo cameras (e.g., a left camera and a right camera) to provide the doctors various views of surgical sites during the operations. The images from the cameras are also used to take measurements in the surgical sites (e.g., measuring depth or distance to an anatomical structure). Due to temperature, pressure, and/or other conditions that exist when operating an endoscope, the cameras may warp or drift, which introduces misalignment between the images from the cameras. As a result, the measurements taken using the images from the cameras becomes inaccurate.

### SUMMARY

The present disclosure describes a system and method for camera calibration. According to an embodiment, a system includes a memory and a controller communicatively coupled to the memory. The controller moves an endoscope through a cannula and stops the endoscope at a position in the cannula. The controller also captures, using the endoscope at the position, images of a reference corresponding to the cannula and adjusts a parameter of the endoscope based on the reference in the images.

According to another embodiment, a method includes moving an endoscope through a cannula and stopping the endoscope at a position in the cannula. The method also includes capturing, using the endoscope at the position, images of a reference corresponding to the cannula and adjusting a parameter of the endoscope based on the reference in the images. Other embodiments include a non-transitory machine-readable medium storing instructions that, when executed by a processor, cause the processor to perform the method.

The foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

FIGS. 1A through 1C illustrate an example medical system.

FIG. 2A illustrates an example medical system.

FIG. 2B illustrates an example medical instrument system in the medical system of FIG. 2A.

FIG. 2C illustrates an example portion of the medical instrument system of FIG. 2B.

FIG. 3 illustrates an example operation for taking measurements.

FIGS. 4A through 4D illustrate example operations for adjusting an endoscope camera.

FIG. 5 illustrates an example operation for adjusting an endoscope camera.

FIGS. 6A through 6C illustrate example operations for determining camera misalignment.

FIG. 7 is a flowchart of an example method for adjusting an endoscope camera.

### DETAILED DESCRIPTION

Doctors use computer assisted medical systems to perform medical tasks. For example, doctors may use computer assisted surgical systems to perform operations on patients, even remotely. These surgical systems may use endoscopes with stereo cameras to provide the doctors various views of surgical sites during the operations (e.g., by a camera capturing a video of the surgical site). Images from the endoscopes may also be used to take measurements in the surgical sites. For example, a digital ruler application may use the images from the endoscope to measure the distances between points in the surgical sites or distances to points in the surgical sites. As another example, during a fluorescence imaging procedure, a fluorescent dye or different fluorescent dyes may be injected into tissue. Different depths of the tissue may receive different dyes or different amounts of dyes, which results in the different depts. of the tissue illuminating differently (e.g., different colors, different shades or tones, etc.). A digital ruler application or fluorescent imaging application may use the images of the illuminated tissue captured by the endoscope to measure the depth of various parts of the tissue with similar appearance.

As the endoscopes are operated, temperature, pressure, and other environmental conditions may cause the camera lenses of the endoscope to drift, which introduces misalignment into the stereo images from the cameras. The misalignment may cause certain points in the images to move by a small number of pixels. This misalignment may be small and easy to miss, but the misalignment may cause the measurements taken using the images to become inaccurate. For example, when the existing parameters of the endoscope (e.g., extrinsic parameters) are used to convert the pixel coordinates of certain points in the images into three-dimensional coordinates in the global space, the misalignment may cause the three-dimensional coordinates to include error or inaccuracies. These inaccurate measurements make it more difficult for the doctors to operate safely in the surgical site. For example, an inaccurate distance or depth measurement between points at a surgical site may cause a surgeon to move a surgical tool too far, which may cause the tool to make a larger cut or incision than necessary.

The present disclosure describes a medical system (e.g., a surgical system) that detects misalignment between the cameras of an endoscope and adjusts parameters (e.g., camera calibration parameters) of the endoscope to address the misalignment. Generally, when the endoscope is sent through a cannula (e.g., a tube) towards a surgical site, the system stops the endoscope at a position in the cannula (e.g., at a first marking printed on the inside of the cannula, at a predetermined stop location in the cannula, etc.). The endoscope captures images of a reference corresponding to the cannula (e.g., a second marking with a known size printed on the inside of the cannula a predetermined distance away from the first marking, an opening at the end of the cannula, etc.) using the cameras of the endoscope. The system analyzes the images to determine pixel misalignments between the images. Because the system knows the size of the reference, the system may calculate distance misalignments from the pixel misalignments. The system then adjusts parameters of the endoscope (e.g., extrinsic parameters) to address or compensate for the distance misalignments.

In certain embodiments, the medical system provides several technical advantages. For example, by adjusting the parameters of the endoscope, the system compensates for the distance misalignments, which allows images from the endoscope to be used to make measurements (e.g., depth measurements, distance measurements, etc.) at the surgical site. As another example, the system improves the accuracy of the measurements made using the images from the endoscope, which improves patient health and safety.

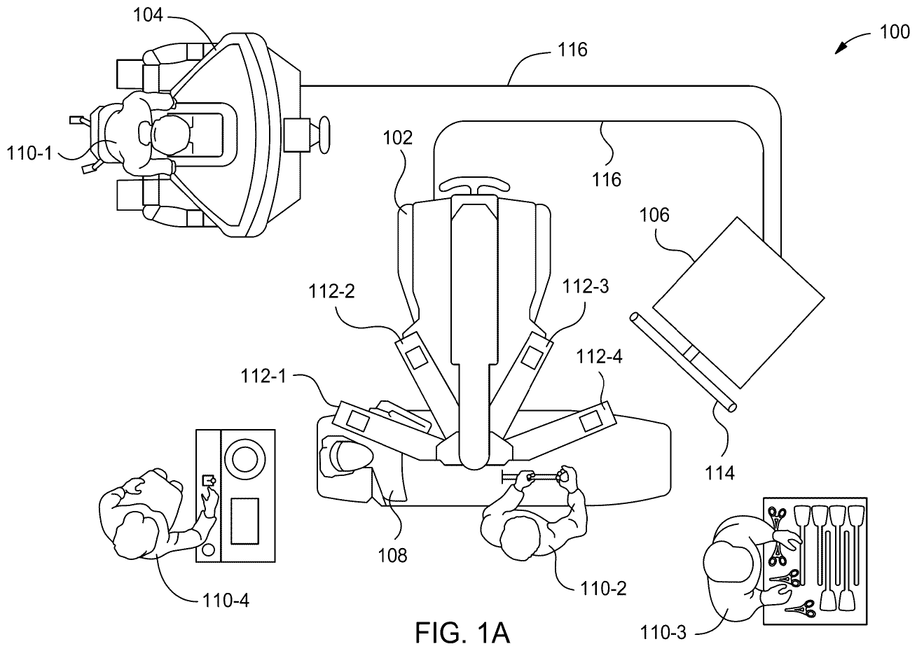

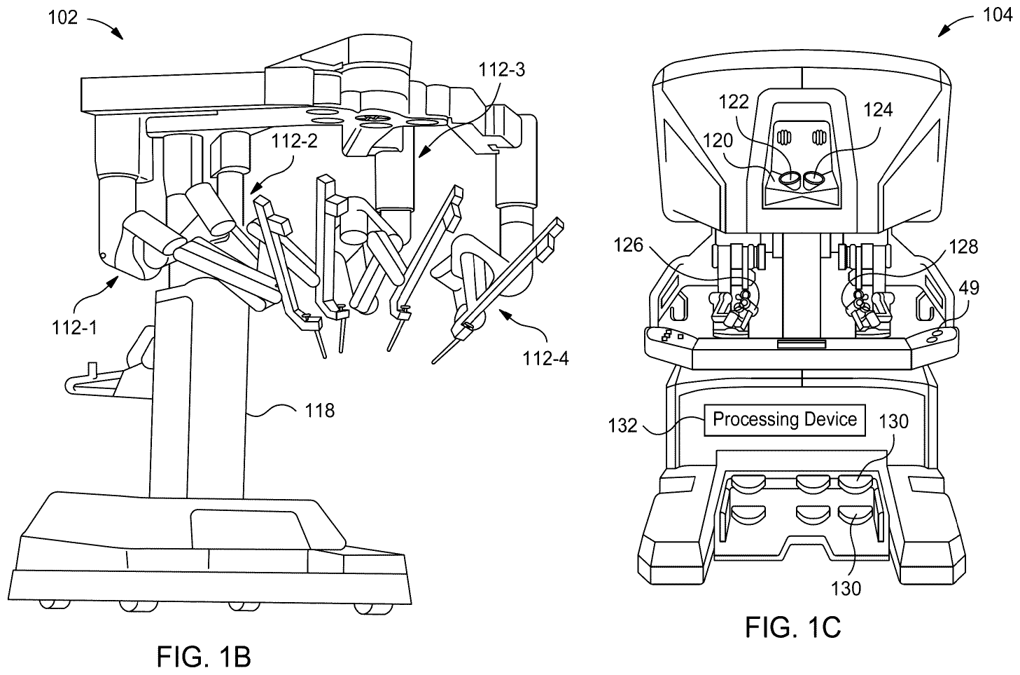

In some examples, one or more components of the medical system may be implemented as a computer-assisted surgical system. It is understood, however, that the medical system may be implemented in any type of medical system (e.g., digital fiducial systems, anatomy detection systems, and clinical guidance systems). FIG. 1A shows an example computer-assisted surgical system 100 that may implement some of the features described herein.

The surgical system 100 may include a manipulator assembly 102, a user control apparatus 104, and an auxiliary apparatus 106, all of which are communicatively coupled to each other. The surgical system 100 may be utilized by a medical team to perform a computer-assisted medical procedure or other similar operation on a body of a patient 108 or on any other body as may serve a particular implementation. The medical team may include a first user 110-1 (such as a surgeon for a surgical procedure), a second user 110-2 (such as a patient-side assistant), a third user 110-3 (such as another assistant, a nurse, a trainee, etc.), and a fourth user 110-4 (such as an anesthesiologist for a surgical procedure), all of whom may be collectively referred to as users 110, and each of whom may control, interact with, or otherwise be a user of the surgical system 100. More, fewer, or alternative users may be present during a medical procedure as may serve a particular implementation. For example, team composition for different medical procedures, or for non-medical procedures, may differ and include users with different roles.

Although FIG. 1A illustrates an ongoing minimally invasive medical procedure such as a minimally invasive surgical procedure, it will be understood that the surgical system 100 may similarly be used to perform open medical procedures or other types of operations. For example, operations such as exploratory imaging operations, mock medical procedures used for training purposes, and/or other operations may also be performed.

The manipulator assembly 102 may include one or more manipulator arms 112 (e.g., manipulator arms 112-1 through 112-4) to which one or more instruments may be coupled. The instruments may be used for a computer-assisted surgical procedure on the patient 108 (e.g., by being at least partially inserted into the patient 108 and manipulated within the patient 108). While the manipulator assembly 102 is depicted and described herein as including four manipulator arms 112, the manipulator assembly 102 may include a single manipulator arm 112 or any other number of manipulator arms as may serve a particular implementation. Although the example of FIG. 1A illustrates the manipulator arms 112 as robotic manipulator arms, in some examples, one or more instruments may be partially or entirely manually controlled, such as by being handheld and controlled manually by a person. These partially or entirely manually controlled instruments may be used in conjunction with, or as an alternative to, computer-assisted instrumentation that is coupled to the manipulator arms 112.

During the medical operation, the user control apparatus 104 may facilitate teleoperational control by the user 110-1 of the manipulator arms 112 and instruments attached to the manipulator arms 112. To this end, the user control apparatus 104 may provide the user 110-1 with imagery of an operational area associated with the patient 108 as captured by an imaging device. The manipulator arms 112 or any instruments coupled to the manipulator arms 112 may mimic the dexterity of the hand, wrist, and fingers of the user 110-1 across multiple degrees of freedom of motion. In this manner, the user 110-1 may intuitively perform a procedure (e.g., an incision procedure, a suturing procedure, etc.) using one or more of the manipulator arms 112 or any instruments coupled to the manipulator arms 112.

The auxiliary apparatus 106 may include one or more computing devices that perform auxiliary functions in support of the procedure, such as providing insufflation, electrocautery energy, illumination or other energy for imaging devices, image processing, or coordinating components of the surgical system 100. In some examples, the auxiliary apparatus 106 may include a display monitor 114 that displays one or more user interfaces, or graphical or textual information in support of the procedure. In some instances, the display monitor 114 may be a touchscreen display that provides user input functionality. Augmented content provided by a region-based augmentation system may be similar, or differ from, content associated with the display monitor 114 or one or more display devices in the operation area (not shown).

The manipulator assembly 102, user control apparatus 104, and auxiliary apparatus 106 may be communicatively coupled one to another in any suitable manner. For example, the manipulator assembly 102, user control apparatus 104, and auxiliary apparatus 106 may be communicatively coupled by way of control lines 116, which may represent any wired or wireless communication link as may serve a particular implementation. To this end, manipulator assembly 102, user control apparatus 104, and auxiliary apparatus 106 may each include one or more wired or wireless communication interfaces, such as one or more local area network interfaces, Wi-Fi network interfaces, cellular interfaces, and so forth.

FIG. 1B illustrates an example manipulator assembly 102. As seen in FIG. 1B, the manipulator assembly 102 includes a base 118, a manipulator arm 112-1, a manipulator arm 112-2, a manipulator arm 112-3, and a manipulator arm 112-4. Each manipulator arm 112-1, 112-2, 112-3, and 112-4 is pivotably coupled to the base 118. Although the base 118 may include casters to allow ease of mobility, in some embodiments, the manipulator assembly 102 is fixedly mounted to a floor, ceiling, operating table, structural framework, or the like.

In a typical procedure, two of the manipulator arms 112-1, 112-2, 112-3, or 112-4 hold surgical instruments and a third holds a stereo endoscope. The remaining manipulator arms are available so that other instruments may be introduced at the work site. Alternatively, the remaining manipulator arms may be used for introducing another endoscope or another image capturing device, such as an ultrasound transducer, to the work site.

Each of the manipulator arms 112-1, 112-2, 112-3, and 112-4 may be formed of links that are coupled together and manipulated through actuatable joints. Each of the manipulator arms 112-1, 112-2, 112-3, and 112-4 may include a setup arm and a device manipulator. The setup arm positions its held device so that a pivot point occurs at its entry aperture into the patient. The device manipulator may then manipulate its held device so that the held device may be pivoted about the pivot point, inserted into and retracted out of the entry aperture, and rotated about its shaft axis. Each of the manipulator arms 112-1, 112-2, 112-3, and 112-4 may include sensors (e.g., kinematics sensors, position sensors, accelerometers, etc.) that detect or track movement of the manipulator arms 112-1, 112-2, 112-3, and 112-4. For example, these sensors may detect how far or how quickly a manipulator arm 112-1, 112-2, 112-3, or 112-4 moves in a certain direction.

FIG. 1C illustrates an example user control apparatus 104. The user control apparatus 104 includes a stereo vision display 120 so that the user may view the surgical work site in stereo vision from images captured by the stereoscopic camera of the manipulator assembly 102. Left and right eyepieces 122 and 124 are provided in the stereo vision display 120 so that the user may view left and right display screens inside the display 120 respectively with the user's left and right eyes. While viewing typically an image of the surgical site on a suitable viewer or display, the surgeon performs the surgical procedures on the patient by manipulating master control input devices, which in turn control the motion of robotic instruments.

The user control apparatus 104 also includes left and right input devices 126 and 128 that the user may grasp respectively with his/her left and right hands to manipulate devices (e.g., surgical instruments) being held by the manipulator arms 112-1, 112-2, 112-3, and 112-3 of the manipulator assembly 102 in preferably six or more degrees of freedom (“DOF”). Foot pedals 130 with toe and heel controls are provided on the user control apparatus 104 so the user may control movement and/or actuation of devices associated with the foot pedals.

A processing device 132 is provided in the user control apparatus 104 for control and other purposes. The processing device 132 performs various functions in the surgical system 100. One function performed by processing device 132 may be to translate and transfer the mechanical motion of input devices 126 and 128 to actuate their corresponding joints in their associated manipulator arms 112-1, 112-2, 112-3, and 112-4 so that the surgeon can effectively manipulate devices, such as the surgical instruments. Another function of the processing device 132 may be to implement the methods, crosscoupling control logic, and controllers or processors described herein. The auxiliary apparatus 106 may include a processing device 132 that performs the functions or actions described herein. The processing device 132 may include a processor and a memory that perform the functions described herein.

The processor may include any electronic circuitry, including, but not limited to one or a combination of microprocessors, microcontrollers, application specific integrated circuits (ASIC), application specific instruction set processor (ASIP), and/or state machines, that communicatively couples to a memory and controls the operation of the user control apparatus 104 and/or the auxiliary apparatus 106. The processor may be 8-bit, 16-bit, 32-bit, 64-bit or of any other suitable architecture. The processor may include an arithmetic logic unit (ALU) for performing arithmetic and logic operations, processor registers that supply operands to the ALU and store the results of ALU operations, and a control unit that fetches instructions from memory and executes them by directing the coordinated operations of the ALU, registers and other components. The processor may include other hardware that operates software to control and process information. The processor executes software stored on a memory to perform any of the functions described herein. The processor controls the operation and administration of the user control apparatus 104 or the auxiliary apparatus 106 by processing information (e.g., information received from the user control apparatus 104, the manipulator assembly 102, the auxiliary apparatus 106, and/or a memory). The processor is not limited to a single processing device and may encompass multiple processing devices contained in the same device or computer or distributed across multiple devices or computers. The processor is considered to perform a set of functions or actions if the multiple processing devices collectively perform the set of functions or actions, even if different processing devices perform different functions or actions in the set.

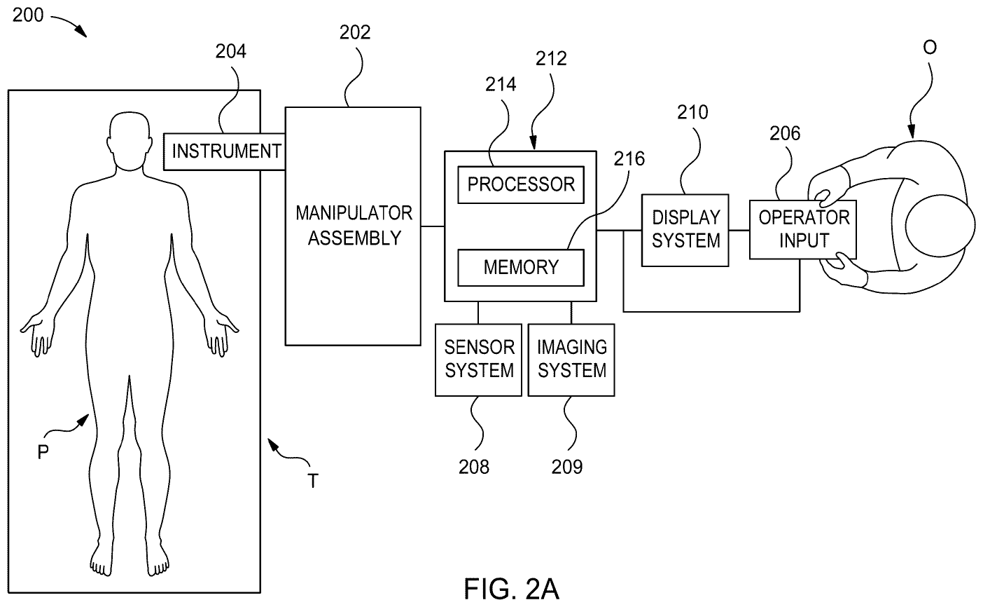

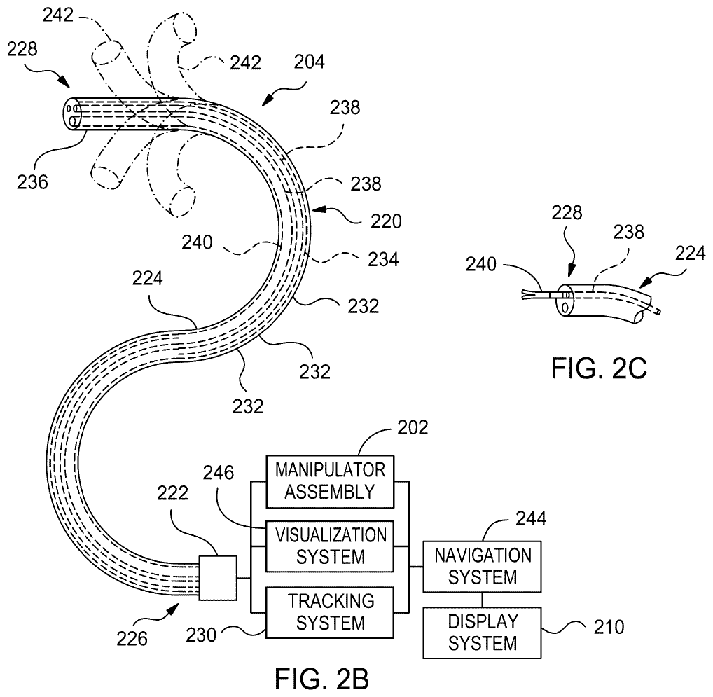

FIG. 2A illustrates an example computer-assisted surgical system 200 that may implement some of the features described herein. The surgical system 200 can be used, for example, in surgical, diagnostic, therapeutic, biopsy, or non-medical procedures. As shown in FIG. 2A, the surgical system 200 (which may be a robotically-assisted surgical system) includes one or more manipulator assemblies 202 for operating one or more medical instrument systems 204 in performing various procedures on a patient P positioned on a table T in a medical environment. For example, the manipulator assembly 202 can drive catheter or end effector motion, can apply treatment to target tissue, and/or can manipulate control members. The manipulator assembly 202 can be teleoperated, non-teleoperated, or a hybrid teleoperated and non-teleoperated assembly with select degrees of freedom of motion that can be motorized and/or teleoperated and select degrees of freedom of motion that can be non-motorized and/or non-teleoperated. An operator input system 206, which can be inside or outside of the medical environment, generally includes one or more control devices for controlling the manipulator assembly 202. The manipulator assembly 202 supports a medical instrument system 204 and can optionally include a plurality of actuators or motors that drive inputs on the medical instrument system 204 in response to commands from a control system 212. The actuators can optionally include drive systems that when coupled to the medical instrument system 204 can advance the medical instrument system 204 into a natural or surgically created anatomic orifice. Other drive systems can move the distal end of the medical instrument in multiple degrees of freedom, which can include three degrees of linear motion (e.g., linear motion along the x, y, and z Cartesian axes) and in three degrees of rotational motion (e.g., rotation about the x, y, and z Cartesian axes). The manipulator assembly 202 can support various other systems for irrigation, treatment, or other purposes. Such systems can include fluid systems (e.g., reservoirs, heating/cooling elements, pumps, and valves), generators, lasers, interrogators, and ablation components.

The surgical system 200 also includes a display system 210 for displaying an image or representation of the surgical site and a medical instrument system 204. The image or representation is generated by an imaging system 209, which may include an endoscopic imaging system. The display system 210 and operator input system 206 may be oriented so that an operator O can control the medical instrument system 204 and the operator input system 206 with the perception of telepresence. A graphical user interface can be displayable on the display system 210 and/or a display system of an independent planning workstation.

In some examples, the imaging system 209 includes an endoscopic imaging system with components that are integrally or removably coupled to the medical instrument system 204. However, in some examples, a separate imaging device, such as an endoscope, attached to a separate manipulator assembly can be used with the medical instrument system 204 to image the surgical site. The imaging system 209 can be implemented as hardware, firmware, software, or a combination thereof, which interact with or are otherwise executed by one or more computer processors, which can include the processors 214 of the control system 212.

The surgical system 200 also includes a sensor system 208. The sensor system 208 may include a position/location sensor system (e.g., an actuator encoder or an electromagnetic (EM) sensor system) and/or a shape sensor system (e.g., an optical fiber shape sensor) for determining the position, orientation, speed, velocity, pose, and/or shape of the medical instrument system 204. These sensors may also detect a position, orientation, or pose of the patient P on the table T. For example, the sensors may detect whether the patient P is face-down or face-up. As another example, the sensors may detect a direction in which the head of the patient P is directed. The sensor system 208 can also include temperature, pressure, force, or contact sensors, or the like.

The surgical system 200 can also include a control system 212, which includes at least one memory 216 and at least one computer processor 214 for effecting control between the medical instrument system 204, the operator input system 206, the sensor system 208, and the display system 210. The control system 212 includes programmed instructions (e.g., a non-transitory machine-readable medium storing the instructions) to implement a procedure using the surgical system 200, including for navigation, steering, imaging, engagement feature deployment or retraction, applying treatment to target tissue (e.g., via the application of energy), or the like.

The control system 212 may further include a virtual visualization system to provide navigation assistance to the operator O when controlling medical instrument system 204 during an image-guided surgical procedure. Virtual navigation using the virtual visualization system can be based upon reference to an acquired pre-operative or intra-operative dataset of anatomic passageways. The virtual visualization system processes images of the surgical site imaged using imaging technology, such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The control system 212 uses a pre-operative image to locate the target tissue (using vision imaging techniques and/or by receiving user input) and create a pre-operative plan, including an optimal first location for performing treatment. The pre-operative plan can include, for example, a planned size to expand an expandable device, a treatment duration, a treatment temperature, and/or multiple deployment locations.

The processor 214 is any electronic circuitry, including, but not limited to one or a combination of microprocessors, microcontrollers, application specific integrated circuits (ASIC), application specific instruction set processor (ASIP), and/or state machines, that communicatively couples to the memory 216 and controls the operation of the control system 212. The processor 214 may be 8-bit, 16-bit, 32-bit, 64-bit or of any other suitable architecture. The processor 214 may include an arithmetic logic unit (ALU) for performing arithmetic and logic operations, processor registers that supply operands to the ALU and store the results of ALU operations, and a control unit that fetches instructions from memory and executes them by directing the coordinated operations of the ALU, registers and other components. The processor 214 may include other hardware that operates software to control and process information. The processor 214 executes software stored on the memory 216 to perform any of the functions described herein. The processor 214 controls the operation and administration of the control system 212 by processing information (e.g., information received from the manipulator assembly 202, the operator input system 206, and the memory 216). The processor 214 is not limited to a single processing device and may encompass multiple processing devices contained in the same device or computer or distributed across multiple devices or computers. The processor 214 is considered to perform a set of functions or actions if the multiple processing devices collectively perform the set of functions or actions, even if different processing devices perform different functions or actions in the set.

The memory 216 may store, either permanently or temporarily, data, operational software, or other information for the processor 214. The memory 216 may include any one or a combination of volatile or non-volatile local or remote devices suitable for storing information. For example, the memory 216 may include random access memory (RAM), read only memory (ROM), magnetic storage devices, optical storage devices, or any other suitable information storage device or a combination of these devices. The software represents any suitable set of instructions, logic, or code embodied in a computer-readable storage medium. For example, the software may be embodied in the memory 216, a disk, a CD, or a flash drive. In particular embodiments, the software may include an application executable by the processor 214 to perform one or more of the functions described herein. The memory 216 is not limited to a single memory and may encompass multiple memories contained in the same device or computer or distributed across multiple devices or computers. The memory 216 is considered to store a set of data, operational software, or information if the multiple memories collectively store the set of data, operational software, or information, even if different memories store different portions of the data, operational software, or information in the set.

FIG. 2B illustrates an example medical instrument system 204 in the surgical system 200. In some embodiments, the medical instrument system 204 is used in an image-guided medical procedure. For example, the medical instrument system 204 may be used for non-teleoperational exploratory procedures or in procedures involving traditional manually operated medical instruments, such as endoscopy.

The medical instrument system 204 includes an elongate flexible device 220, such as a flexible catheter or endoscope (e.g., gastroscope, bronchoscope), coupled to a drive unit 222. The elongate flexible device 220 includes a flexible body 224 having a proximal end 226 and a distal end, or tip portion, 228. In some embodiments, the flexible body 224 has an approximately 14-20 millimeter outer diameter. Other flexible body outer diameters may be larger or smaller. The flexible body 224 has an appropriate length to reach certain portions of the anatomy, such as the lungs, sinuses, throat, or the upper or lower gastrointestional region, when the flexible body 224 is inserted into a patient's oral or nasal cavity.

The medical instrument system 204 includes a tracking system 230 for determining the position, orientation, speed, velocity, pose, and/or shape of the distal end 228 and/or of one or more segments 232 along the flexible body 224 using one or more sensors and/or imaging devices. The entire length of the flexible body 224, between the distal end 228 and the proximal end 226, is effectively divided into the segments 232. The tracking system 230 is implemented as hardware, firmware, software, or a combination thereof, which interact with or are otherwise executed by one or more computer processors, which may include the processors 214 of control system 212.

The tracking system 230 tracks distal the end 228 and/or one or more of the segments 232 using a shape sensor 234. In some embodiments, the tracking system 230 tracks the distal end 228 using a position sensor system 236, such as an electromagnetic (EM) sensor system. In some examples, the position sensor system 236 measures six degrees of freedom (e.g., three position coordinates x, y, and z and three orientation angles indicating pitch, yaw, and roll of a base point) or five degrees of freedom (e.g., three position coordinates x, y, and z and two orientation angles indicating pitch and yaw of a base point).

The flexible body 224 includes one or more channels 238 sized and shaped to receive one or more medical instruments 240. In some embodiments, the flexible body 224 includes two channels 238 for separate instruments 240, however, a different number of channels 238 can be provided. FIG. 2C illustrates an example portion of the medical instrument system 204 of FIG. 2B. As seen in FIG. 2C, the medical instrument 240 extends through the flexible body 224. In some embodiments, the medical instrument 240 can be used for procedures and aspects of procedures, such as surgery, biopsy, ablation, mapping, imaging, illumination, irrigation, or suction. The medical instrument 240 is deployed through the channel 238 of the flexible body 224 and is used at a target location within the anatomy. The medical instrument 240 includes, for example, image capture devices, biopsy instruments, ablation instruments, catheters, laser ablation fibers, and/or other surgical, diagnostic, or therapeutic tools. The medical tools include end effectors having a single working member such as a scalpel, a blunt blade, a lens, an optical fiber, an electrode, and/or the like. Other end effectors include, for example, forceps, graspers, balloons, needles, scissors, clip appliers, and/or the like. Other end effectors further include electrically activated end effectors such as electrosurgical electrodes, transducers, sensors, imaging devices, and/or the like. The medical instrument 240 is advanced from the opening of the channel 238 to perform the procedure and then retracted back into the channel when the procedure is complete. The medical instrument 240 is removed from the proximal end 226 of the flexible body 224 or from another optional instrument port (not shown) along the flexible body 224. The medical instrument 240 may be used with an image capture device (e.g., an endoscopic camera) also within the elongate flexible device 220. Alternatively, the medical instrument 240 may itself be the image capture device.

The medical instrument 240 additionally houses cables, linkages, or other actuation controls (not shown) that extend between the proximal and distal ends to controllably bend the distal end of the medical instrument 240. The flexible body 224 also houses cables, linkages, or other steering controls (not shown) that extend between the drive unit 222 and the distal end 228 to controllably bend the distal end 228 as shown, for example, by the broken dashed line depictions 242 of the distal end 228. In some examples, at least four cables are used to provide independent “up-down” steering to control a pitch motion of the distal end 228 and “left-right” steering to control a yaw motion of the distal end 228. In embodiments in which the medical instrument system 204 is actuated by a robotically-assisted assembly, the drive unit 222 can include drive inputs that removably couple to and receive power from drive elements, such as actuators, of the teleoperational assembly. In some embodiments, the medical instrument system 204 includes gripping features, manual actuators, or other components for manually controlling the motion of the medical instrument system 204. The information from the tracking system 230 can be sent to a navigation system 244, where the information is combined with information from the visualization system 246 and/or the preoperatively obtained models to provide the physician or other operator with real-time position information.

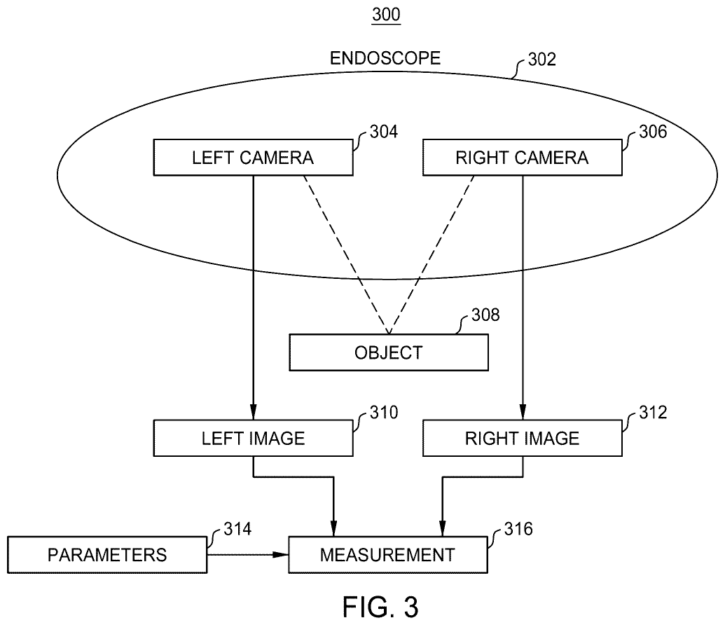

FIGS. 3 through 7 illustrate example operations performed by a computer system in a medical system (e.g., the surgical system 100 of FIG. 1A or the surgical system 200 of FIG. 2A). Generally, the computer system (which may be implemented in the user control apparatus 104 and/or the auxiliary apparatus 106 of the surgical system 100 using the processing device 132 and/or in the control system 212 of the surgical system 200 using the processor 214 and the memory 216) detects misalignments between cameras of an endoscope and adjusts parameters of the endoscope to address or compensate for the misalignments.

The computer system may be described as performing certain actions (e.g., stopping the endoscope or carriage, capturing images, etc.) that may involve other components, such as the endoscope, carriage, endoscope, etc. In these instances, it is understood that the controller performs these actions by communicating signals to the other components that causes those components to perform the actions.

FIG. 3 illustrates an example operation 300 for taking measurements. The computer system performs the operation 300. As seen in FIG. 3, the operation 300 involves an endoscope 302 (which may be capturing images. The endoscope 302 may be a stereo endoscope that includes a left camera 304 and a right camera 306. These cameras 304 and 306 may be offset from each other and may capture images of an object 308. For example, the endoscope 302 may be positioned at a surgical site, and the cameras 304 and 306 may capture images of an anatomical object at the surgical site. In the example of FIG. 3, the left camera 304 captures a left image 310 of the object 308, and the right camera 306 captures a right image 312 of the object 308.

Although the left camera 304 and the right camera 306 are positioned close to each other on the endoscope 302, the left camera 304 and the right camera 306 are at different physical positions and have different orientations. As a result, the left camera 304 and the right camera 306 capture different images of the object 308. For example, the left image 310 and the right image 312 may show different perspectives of the object 308. A portion of the object 308 may appear in a certain set of pixels in the left image 310, and the same portion of the object 308 may appear in a slightly different set of pixels in the right image 312.

The computer system may use the left image 310, the right image 312, and parameters 314 to determine measurements 316 at the surgical site. For example, the computer system may measure depth and/or distance using the left image 310, the right image 312, and the parameters 314. The parameters 314 may include extrinsic parameters (e.g., extrinsic camera calibration parameters) and intrinsic parameters (e.g., intrinsic camera calibration parameters) of the endoscope 302. The extrinsic parameters may indicate the pose of the left camera 304 and the pose of the right camera 306. For example, the extrinsic parameters may include a rotation matrix and/or a translation vector that indicate the position and/or orientation of the left camera 304 and/or the right camera 306. The intrinsic parameters may indicate how the left camera 304 and the right camera 306 capture the left image 310 and the right image 312. For example, the intrinsic parameters may include an optical axis, focal length, principal point, skew coefficients, etc.

The computer system uses the parameters 314 to determine the measurement 316 from the left image 310 and the right image 312. For example, the computer system may use the intrinsic parameters and extrinsic parameters to convert the pixels in the two-dimensional (2D) planes of the left image 310 and/or the right image 312 into three-dimensional (3D) coordinates in the world. The computer system may then use the 3D coordinates to determine measurements 316, such as depths and distances.

As discussed previously, during operation of the endoscope 302 at different surgical sites, the endoscope 302 may be subject to various temperatures and/or pressures. These temperatures and pressures may cause shifting or other physical distortions to the left camera 304 and/or the right camera 306. As a result of these distortions, the left image 310 and/or the right image 312 produced by the left camera 304 and/or the right camera 306 are also distorted. For example, the object 308 in the left image 310 and/or the right image 312 may occupy a different set of pixels in the left image 310 and/or the right image 312, which may lead to inaccuracies in the measurement 316. When the computer system uses the left image 310 and the right image 312 to calculate the measurement 316, the shift and/or distortion in the left image 310 and/or the right image 312 may cause the computer system to calculate a depth and/or a distance that is greater or less than the actual depth or distance. When a user moves a surgical tool at the surgical site based on the inaccurate depth and/or distance, it may cause damage or injury at the surgical site.

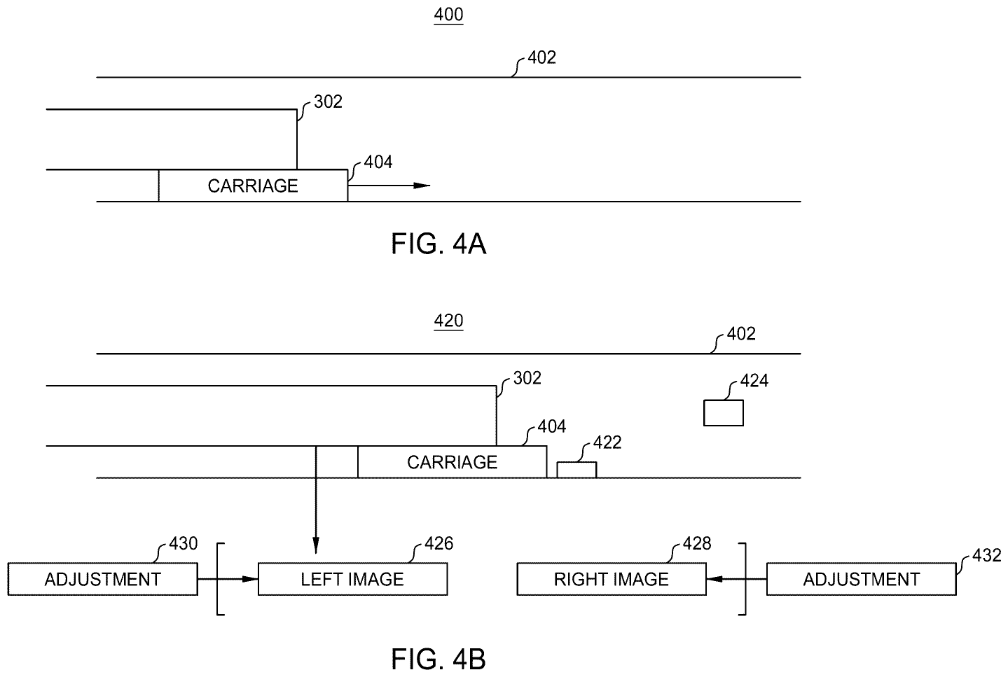

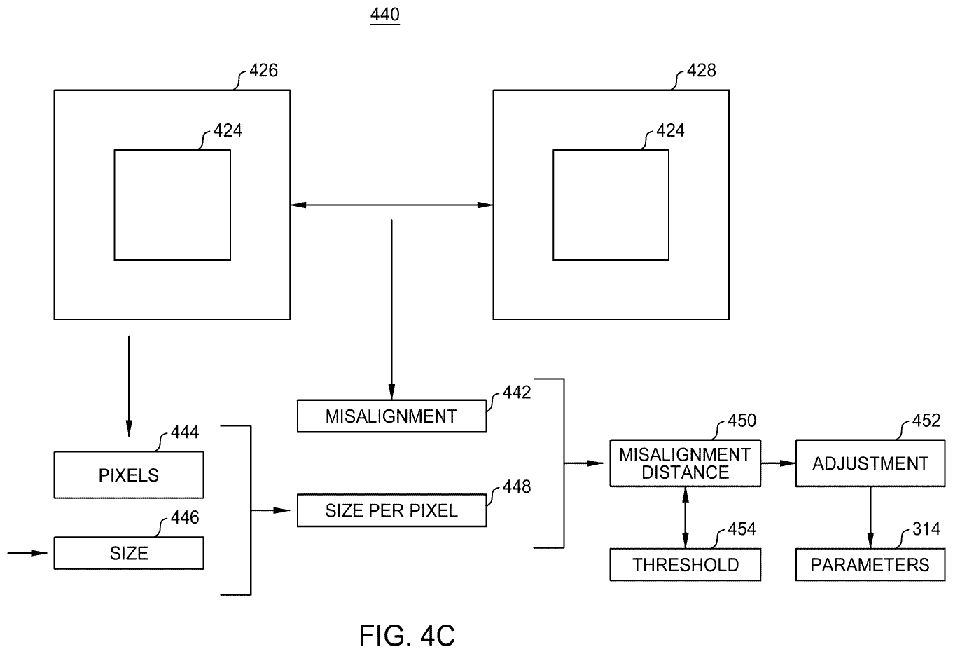

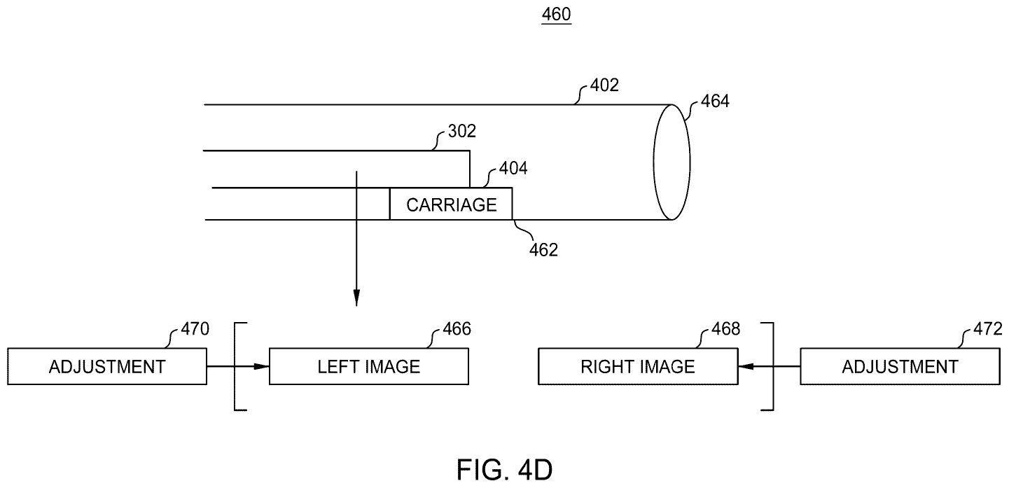

FIGS. 4A through 4D illustrate example operations for adjusting an endoscope camera. Generally, the computer system performs these operations to determine distortions in the cameras of the endoscope before the endoscope reaches the surgical site. The computer system may then adjust the parameters of the endoscope to account for these distortions. When the computer system subsequently uses the adjusted parameters to calculate measurements (e.g., distances, depths, etc.) at the surgical site from the images captured by the endoscope, the computer system produces more accurate measurements, which may improve patient health.

FIG. 4A illustrates an example operation 400 performed by the computer system to move the endoscope 302 through a cannula 402. Generally, the computer system navigates the endoscope 302 towards a surgical site by moving the endoscope 302 through a cannula 402, which may resemble a tube. An operator may position the endoscope 302 on a carriage 404 positioned in the cannula 402. The computer system may then move the carriage 404 through the cannula 402 to move the endoscope 302 through the cannula 402. When the endoscope 302 reaches the end of the cannula 402, the endoscope 302 may emerge from the cannula 402 into the surgical site.

FIG. 4B illustrates an example operation 420 performed by the computer system to calibrate the endoscope 302. The cannula 402 has markings printed on an inside wall of the cannula 402. As the endoscope 302 moves on the carriage 404 through the cannula 402, the endoscope 302 may encounter these markings. In the example of FIG. 4B, the endoscope 302 encounters a first marking 422 printed on the inner wall of the cannula 402. Additionally, a second marking 424 (e.g., a square or rectangle, an arrangement of dots, an April tag, etc.), which serves as a reference, is printed on the inner wall of the cannula 402 a predetermined or preset distance away from the first marking 422. Generally, the second marking 424 may be any visual tag that can provide subpixel coordination in the image space. When the endoscope 302 encounters the first marking 422, the computer system may stop the carriage 404 and/or the endoscope 302 to prevent the endoscope 302 from moving further down the cannula 402. For example, when the computer system detects the first marking 422 at a particular position in a video or image captured by the endoscope 302, the computer system may stop the carriage 404 and/or the endoscope 302. In this manner, the computer system stops the endoscope 302 a predetermined or preset distance away from the second marking 424.

In some embodiments, multiple second markings 424 are printed on the inner wall of the cannula 402. For example, the second markings 424 (e.g., multiple April tags) may be printed to form a ring on the inner wall of the cannula 402. Additionally, any type of ink may be used to print the second marking 424. For example, an ultraviolet marking trail ink may be used to print the second marking 424.

The computer system then uses the endoscope 302 to capture images of the second marking 424 from the predetermined or preset distance. In the example of FIG. 4B, the computer system uses the cameras of the endoscope 302 to capture a left image 426 and a right image 428 of the second marking 424. The left image 426 may be captured by a left camera of the endoscope 302, and the right image 428 may be captured by a right camera of the endoscope 302.

The computer system then makes an adjustment 430 to the left image 426 and an adjustment 432 to the right image 428. The adjustments 430 and 432 may dewarp the left image 426 and the right image 428. For example, due to the shape of the lenses on the left camera and the right camera, the second marking 424 shown in the left image 426 and the right image 428 may include distortions that introduce additional curvature. The adjustments 430 and 432 may remove some of this curvature, which straightens lines and produces more accurate depictions of the second marking 424. In some embodiments, the adjustments 430 and 432 may also shift or move the second marking 424 in the left image 426 and/or the right image 428 to account for the different positions and/or orientations of the left camera and the right camera. In this manner, the computer system brings the left image 426 and the right image 428 into the same image plane.

As an example, the computer system may detect the edges and/or corners of the second marking 424 in the left image 426 and the right image 428. The computer system then straightens the edges and/or corners of the second marking 424. In some instances, the computer system may not dewarp or straighten the other portions of the second marking 424.

In embodiments where multiple second markings 424 are printed on the inner wall of the cannula, the left image 426 and the right image 428 may show multiple second markings 424. The computer system may detect the edges and/or corners of these second markings 424 in the left image 426 and the right image 428. These edges and/or corners may provide sufficient information for the computer system to calibrate the endoscope.

FIG. 4C illustrates an example operation 440 performed by the computer system to adjust the parameters of the endoscope. The computer system begins with the left image 426 and the right image 428 of the second marking 424 after adjusting the left image 426 and the right image 428. The computer system compares the left image 426 and the right image 428 to determine a misalignment 442 between the left image 426 and the right image 428. For example, the computer system may determine the pixels 444 in the left image 426 and the right image 428 occupied by the second marking 424. The computer system may compare these pixels 444 to determine a difference in the pixels 444 occupied by the second marking 424 between the left image 426 and the right image 428. The misalignment 442 may indicate this difference. For example, the misalignment 442 may indicate a horizontal difference in the pixels 444 and a vertical difference in the pixels 444. Thus, the misalignment 442 may indicate a number of pixels 444 that the second marking 424 in the left image 426 is offset from the second marking 424 in the right image 428.

In some embodiments, the computer system converts the misalignment 442 into a translational misalignment and a rotational misalignment. The translational misalignment may be a translational component of the misalignment 442, and the rotational misalignment may be a rotational component of the misalignment 442. For example, the translational component may indicate magnitude (e.g., measured in pixels) of the misalignment 442 along a directional axis of the image space (e.g., along a horizontal or vertical axis of the image space). The rotational component may indicate an angular component of the misalignment 442 (e.g., an angular offset from the horizontal or vertical axis of the image space).

The computer system is also provided a size 446 of the second marking 424. For example, the size 446 may be a parameter or input that is provided to the computer system when operating using the cannula. The size 446 may indicate a physical size of the second marking 424 printed in the cannula. For example, the size 446 may indicate the physical dimensions (e.g., length and width) of the second marking 424. The computer system calculates a size per pixel 448 using the size 446 and the pixels 444. For example, the pixels 444 may indicate a number of pixels in a horizontal direction and a number of pixels in a vertical direction occupied by the second marking 424 in the left image 426 and/or the right image 428. The computer system may divide the size 446 by the number of pixels 444 to determine the size per pixel 448. For example, the computer system may divide a horizontal dimension indicated by the size 446 by the number of pixels in the horizontal direction, and the computer system may divide a vertical dimension indicated by the size 446 by the number of pixels in the vertical direction.

The computer system then determines a misalignment distance 450 using the misalignment 442 and the size per pixel 448. The computer system may multiply the misalignment 442 (which is the pixel misalignment) by the size per pixel 448 to produce the misalignment distance 450. As an example, the computer system may multiply a number of pixels of horizontal misalignment indicated by the misalignment 442 by a horizontal size per pixel indicated by the size per pixel 448 to produce a horizontal misalignment distance, and the computer system may multiply a number of pixels of vertical misalignment indicated by the misalignment 442 by a vertical size per pixel indicated by the size per pixel 448 to produce a vertical misalignment distance. Thus, the misalignment distance 450 is a physical distance represented by the pixel misalignment between the second marking 424 in the left image 426 and the second marking 424 in the right image 428. The size per pixel 448 effectively converts the misalignment 442 in the image or pixel space to the misalignment distance 450 in the world or global space.

In embodiments where the computer system determined the translational misalignment and the rotational misalignment, the computer system may determine the misalignment distance 450 by multiplying the translational misalignment by the size per pixel 448 and by the cosine of the rotational misalignment.

The computer system determines an adjustment 452 to the parameters 314 of the endoscope to correct for the misalignment distance 450. The adjustment 452 may include adjustments to the extrinsic parameters of the endoscope. By adjusting these extrinsic parameters, the computer system calibrates how the 2D pixel coordinates in the images captured by the endoscope are converted to 3D global coordinates to account for or to correct for the misalignment distance 450. In this manner, when the computer system uses the parameters 314 to measure distances or depths from the images captured by the endoscope, the measured distances or depths are accurate and correct for the misalignment distance.

In some embodiments, the computer system compares the misalignment distance 450 to one or more thresholds 454 (e.g., a horizontal threshold and a vertical threshold) to determine whether the computer system should adjust the parameters 314. If the misalignment distance 450 falls below the threshold 454, the computer system may determine that the misalignment distance 450 is within tolerance and maintain the parameters 314. If the misalignment distance 450 exceeds the threshold 454, the computer system may make the adjustment 452 to the parameters 314 to correct for the misalignment distance 450.

In this manner, the computer system adjusts the parameters 314 (e.g., extrinsic parameters) of the endoscope to correct for physical distortions experienced by the cameras of the endoscope. By making these adjustments, the computer system produces more accurate measurements (e.g., distance measurements and/or depth measurements) using the images captured by the endoscope. The more accurate measurements may reduce the chances of injury or harm during a procedure at a surgical site.

In some embodiments, the computer system implements a threshold 454 that indicates whether the endoscope should be used for measuring distances or depths. For example, if the misalignment distance 450 exceeds the threshold 454, the computer system may determine that the endoscope cannot be calibrated to correct the misalignment and that a different endoscope should be used. If the endoscope continues to be used, the computer system may prevent measurement applications (e.g., a digital ruler application or fluorescent imaging application) from loading or being used.

FIG. 4D illustrates an example operation 460 performed by the computer system to calibrate the endoscope 302. Generally, the computer system may perform the operation 460 when markings are unavailable on the inner wall of the cannula.

As seen in FIG. 4D, the carriage 404 may move the endoscope 302 through the cannula 402. The carriage 404 may stop the endoscope 302 at a position 462 in the cannula 402. The position 462 may be a predetermined position that is a predetermined or preset distance away from an end of the cannula 402. In some instances, the position 462 may be specified by software in the computer system (e.g., a software stop), and the software may stop the carriage 404 when the carriage reaches the position 462. There is an opening 464 (e.g., a circular opening) at the end of the cannula 402, which serves as a reference. When the carriage 404 stops at the position 462, the endoscope 302 may be a predetermined or preset distance away from the opening 464.

The computer system then uses the endoscope 302 to capture images of the opening 464 from the predetermined or preset distance. In the example of FIG. 4D, the computer system uses the cameras of the endoscope 302 to capture a left image 466 and a right image 468 of the opening 464. The left image 466 may be captured by a left camera of the endoscope 302, and the right image 468 may be captured by a right camera of the endoscope 302.

The computer system then makes an adjustment 470 to the left image 466 and an adjustment 472 to the right image 468. The adjustments 470 and 472 may dewarp the left image 466 and the right image 468. For example, due to the shape of the lenses on the left camera and the right camera, the opening 464 shown in the left image 466 and the right image 468 may include distortions that introduce additional curvature. The adjustments 470 and 472 may remove some of this curvature, which straightens or smooths the boundary and produces more accurate depictions of the opening 464. In some embodiments, the adjustments 470 and 472 may also shift or move the opening 464 in the left image 466 and/or the right image 468 to account for the different positions and/or orientations of the left camera and the right camera. In this manner, the computer system brings the left image 466 and the right image 468 into the same image plane.

As an example, the computer system may detect the boundary of the opening 464 in the left image 466 and the right image 468. The computer system then smooths the boundary of the opening 464. In some instances, the computer system may not dewarp or smooth the other portions of the opening 464.

The computer system may then perform the operation 440 shown in FIG. 4C using the left image 466 and the right image 468 (e.g., instead of the left image 426 and the right image 428) to adjust the endoscope 302. For example, the computer system may determine a misalignment between the left image 466 and the right image 468. The computer system may then determine an adjustment to the parameters of the endoscope 302 that will reduce or correct the misalignment. The computer system may use predetermined or present distance between the endoscope 302 and the end of the cannula 402 and the size of the opening 464 to determine the adjustment to the parameters. In this manner, the computer system may correct misalignment in the endoscope 302 even when there are no markings on the inner wall of the cannula 402. Instead, the computer system may use the opening 464 at the end of the cannula 402 as a replacement for the markings.

In some embodiments, the computer system may stop the carriage 404 and the endoscope 302 at multiple locations in the cannula 402. Each of the location may be a different distance from the opening 464. For example, the computer system may stop the endoscope 302 at the location 462 and at the end of the cannula 402 itself. The endoscope 302 may then capture the images of the opening 464 at the end of the cannula 402. The computer system may use the multiple sets of images of the opening 464 to calibrate the endoscope 302.

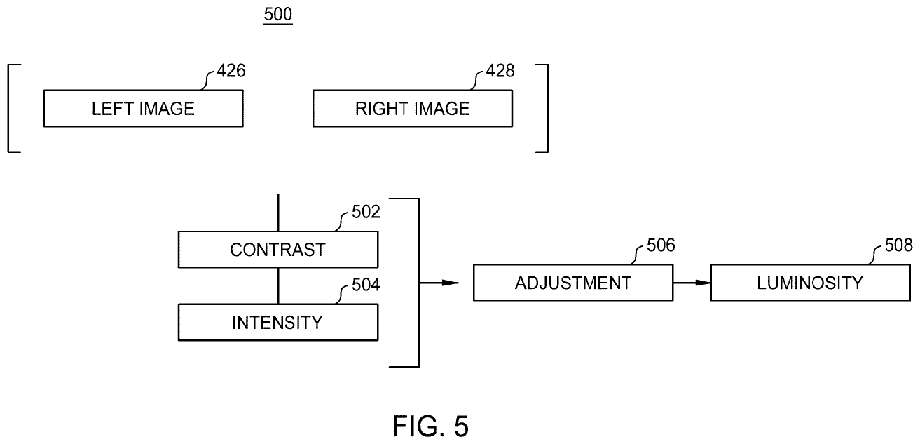

FIG. 5 illustrates an example operation 500 for adjusting an endoscope camera. Generally, the computer system performs the operation 500 to adjust the luminosity of the endoscope. The computer system begins with the left image 426 from the left camera of the endoscope and the right image 428 from the right camera of the endoscope. The computer system analyzes the left image 426 and the right image 428 to determine a contrast 502 and/or an intensity 504 of the left image 426 and the right image 428. The contrast 502 is a metric that indicates a difference in luminance or color that makes an object in the left image 426 and the right image 428 visible against a background of a different luminance or color. The intensity 504 is a metric that indicates the amount of light reflected by an object in the left image 426 and the right image 428. Both of these metrics may indicate how easy it is to discern objects in the left image 426 and the right image 428 and to discern portions of the left image 426 and the right image 428 (e.g., borders of the left image 426 and the right image 428).

The computer system determines an adjustment 506 to a luminosity 508 of the endoscope according to the contrast 502 and the intensity 504. The computer system may compare the contrast 502 and/or intensity 504 to one or more thresholds that indicate whether the luminosity 508 should be increased or decreased. For example, if the contrast 502 and/or the intensity 504 fall below certain thresholds, the computer system may determine the adjustment 506 to increase the luminosity 508. If the contrast 502 and/or the intensity 504 exceed certain thresholds, the computer system may determine the adjustment 506 to decrease the luminosity 508. The magnitude of the adjustment 506 may depend on differences between the contrast 502 and/or the intensity 504 and their respective thresholds. The greater the differences, the greater the magnitude of the adjustment 506, and vice versa.

Adjusting the luminosity 508 of the endoscope may adjust an amount of light emitted by a light (e.g., a light emitting diode) positioned on the endoscope. Increasing the luminosity 508 may increase the amount of light emitted, and decreasing the luminosity 508 may decrease the amount of light emitted. By emitting more or less light, the computer system may increase or decrease the contrast 502 and/or intensity 504 in the images captured by the endoscope. In this manner, the computer system may make it easier to distinguish objects that appear in the images and to distinguish portions of the images from each other.

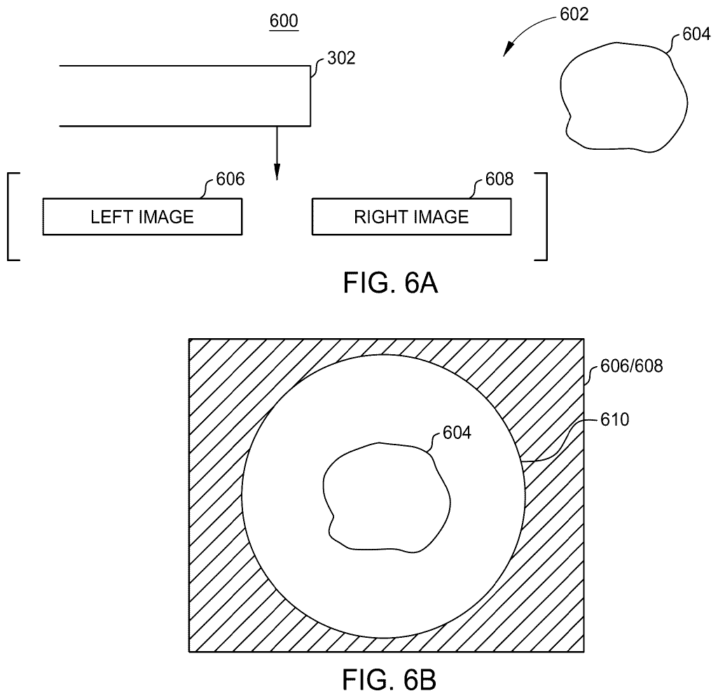

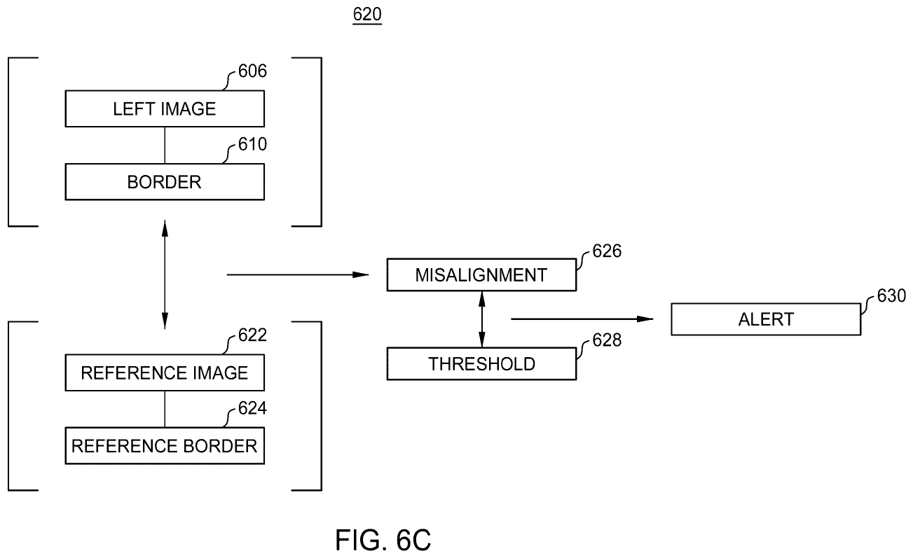

FIGS. 6A through 6C illustrate example operations for determining camera misalignment. Generally, the computer system performs these operations after the endoscope passes through the cannula to the surgical site to determine whether further calibrations of the endoscope should be made.

FIG. 6A shows an example operation 600 performed by the computer system. As seen in FIG. 6A, the endoscope 302 has passed through the cannula to a surgical site 602. An object 604 (e.g., an anatomical object) is located at the surgical site 602. The endoscope 302 is directed towards the object 604. The cameras of the endoscope 302 capture the left image 606 and the right image 608 of the object 604.

FIG. 6B shows an example left image 606 or right image 608. As seen in FIG. 6B, the image 606/608 shows the object 604. Additionally, the image 606/608 includes a border 610 near the periphery of the image. The border 610 may be round, elliptical, or circular depending on properties of the lenses in the cameras of the endoscope. Portions of the image 606/608 between the border 610 and the periphery of the image 606/608 may be black.

FIG. 6C shows an example operation 620 performed by the computer system to determine whether further adjustments are needed to calibrate the endoscope. The computer system begins by analyzing the border 610 in an image from the endoscope (e.g., the left image 606). The computer system compares the image with a reference image 622. Specifically, the computer system compares the border 610 in the image with a reference border 624 in the reference image 622. The reference image 622 may have been captured by the endoscope when the endoscope was confirmed as calibrated. The computer system then stored the reference image 622 for future use.

The computer system compares the border 610 with the reference border 624 to determine a misalignment 626. For example, the computer system may determine whether the border 610 occupies the same pixels in the image as the reference border 624 in the reference image 622. The misalignment 626 may indicate a number of pixels (e.g., a number of pixels in a horizontal direction and/or a number of pixels in a vertical direction) by which the border 610 in the image differs from the reference border 624 in the reference image 622.

The computer system then compares the misalignment 626 with one or more thresholds 628. For example, the computer system may compare the number of pixels in the horizontal direction indicated by the misalignment 626 with a horizontal threshold, and the computer system may compare the number of pixels in the vertical direction indicated by the misalignment 626 with a vertical threshold. If the misalignment 626 falls below the thresholds 628, then the computer system may determine that further calibration of the endoscope is not needed.

If the misalignment 626 exceeds the thresholds 628, then the computer system determines that further calibration should be performed. The computer system generates an alert 630, which may include a message, indicating that further calibration of the endoscope should be performed. The computer system communicates the alert 630 to a user to alert the user about the need for calibration. The user may respond by retracting the endoscope into the cannula to recalibrate the endoscope using the operations shown in FIGS. 4A through 4C.

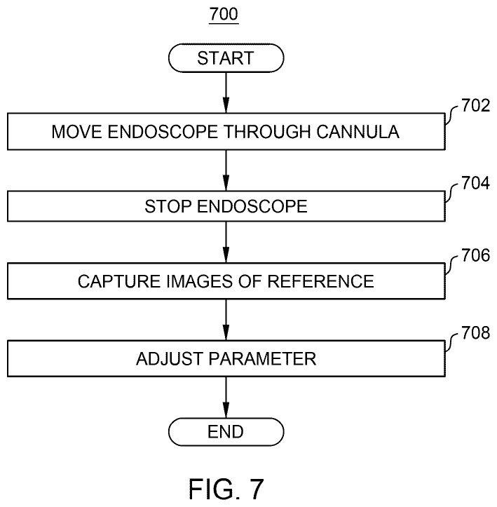

FIG. 7 is a flowchart of an example method 700 for adjusting an endoscope camera. In certain embodiments, a computer system (which may be implemented in the user control apparatus 104 and/or the auxiliary apparatus 106 of the surgical system 100 using the processing device 132 shown in FIGS. 1A through 1C and/or in the control system 212 of the surgical system 200 using the processor 214 and the memory 216 shown in FIGS. 2A through 2C) performs the method 700. By performing the method 700, the computer system calibrates an endoscope.

In block 702, the computer system moves the endoscope through a cannula (which may resemble a tube) towards a surgical site. The cannula may have different markings printed on the inside wall of the cannula. The endoscope may encounter these markings as the endoscope travels through the cannula. The computer system may determine when the endoscope has encountered a marking by detecting the marking in an image or video produced by the endoscope. The computer system may control a carriage on which the endoscope is positioned to move or stop the endoscope in the cannula.

In block 704, the computer system stops the endoscope. For example, the computer system may stop the endoscope according to a first marking. The first marking is printed on the inside wall of the cannula and may indicate a stopping point for the endoscope. For example, the first marking may be a line or box. The computer system may stop the endoscope (e.g., stop the carriage) when the computer system detects, from the image or video from the endoscope, that the endoscope is positioned near or at the first marking. As another example, the computer system may stop the endoscope according to a software stop. The computer system may detect when the endoscope is at a position a predetermined or preset distance away from the end of the cannula and stop the endoscope at the predetermined or preset distance.

In block 706, the computer system uses the endoscope to capture images of a reference. For example, the reference may be a second marking printed on the inside wall of the cannula when the endoscope is stopped. The second marking may be an April tag, and the computer system may know the size (e.g., physical dimensions of the second marking). Additionally, the second marking may be printed a predetermined or preset distance away from the first marking. As another example, the reference may be an opening (e.g., a circular opening) at the end of the cannula. The endoscope may include a stereo camera (e.g., left camera and right camera) that produces multiple images (e.g., left image and right image) of the reference.

In block 708, the computer system adjusts a parameter of the endoscope based on the images of the reference from the endoscope. For example, the computer system may compare the images of the reference (e.g., the left image and the right image) to determine a pixel misalignment between the reference in the images. Because the computer system knows the physical size of the reference, the computer system may use the size of the reference to convert the pixel misalignment into a physical misalignment distance. The computer system then adjusts the parameter of the endoscope to correct for the misalignment distance. After adjusting the parameter, the computer system may then use the parameter to convert 2D coordinates of the pixels in images from the endoscope into 3D global coordinates. The computer then uses the 3D global coordinates to determine measurements (e.g., measured distances and/or depths).

In summary, a medical system (e.g., a surgical system) detects misalignment between the cameras of an endoscope and adjusts parameters of the endoscope to address the misalignment. Generally, when the endoscope is sent through a cannula (e.g., a tube) towards a surgical site, the system stops the endoscope in the cannula. The endoscope captures images of a reference (e.g., a marking on the inner wall of the cannula, an opening at an end of the cannula, etc.) using the cameras of the endoscope. The system analyzes the images to determine pixel misalignments between the images. Because the system knows the size of the reference, the system may calculate distance misalignments from the pixel misalignments. The system then adjusts parameters of the endoscope (e.g., extrinsic parameters) to address or compensate for the distance misalignments.

This description and the accompanying drawings that illustrate aspects, embodiments, or modules should not be taken as limiting. Various mechanical, compositional, structural, electrical, and operational changes may be made without departing from the spirit and scope of this description and the claims. In some instances, well-known circuits, structures, or techniques have not been shown or described in detail in order not to obscure other features. Like numbers in two or more figures represent the same or similar elements.

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Further, the terminology in this description is not intended to be limiting. For example, spatially relative terms-such as “beneath”, “below”, “lower”, “above”, “upper”, “proximal”, “distal”, and the like may be used to describe one element's or feature's relationship to another element or feature as illustrated in the figures. These spatially relative terms are intended to encompass different positions (i.e., locations) and orientations (i.e., rotational placements) of the elements or their operation in addition to the position and orientation shown in the figures. For example, if the content of one of the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly. Likewise, descriptions of movement along and around various axes include various special element positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

Elements described in detail with reference to one embodiment, or module may, whenever practical, be included in other embodiments, or modules in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, or application may be incorporated into other embodiments, or aspects unless specifically described otherwise, unless the one or more elements would make an embodiment or embodiments non-functional, or unless two or more of the elements provide conflicting functions.

In some instances, well known methods, procedures, components, and circuits have not been described in detail so as not to unnecessarily obscure aspects of the embodiments.

This disclosure describes various devices, elements, and portions of computer-assisted devices and elements in terms of their state in three-dimensional space. As used herein, the term “position” refers to the location of an element or a portion of an element in a three-dimensional space (e.g., three degrees of translational freedom along Cartesian x-, y-, and z-coordinates). As used herein, the term “orientation” refers to the rotational placement of an element or a portion of an element (three degrees of rotational freedom—e.g., roll, pitch, and yaw). As used herein, the term “shape” refers to a set positions or orientations measured along an element. As used herein, and for a device with repositionable arms, the term “proximal” refers to a direction toward the base of the computer-assisted device along its kinematic chain and “distal” refers to a direction away from the base along the kinematic chain.

Aspects of this disclosure are described in reference to computer-assisted systems and devices, which may include systems and devices that are teleoperated, remote-controlled, autonomous, semiautonomous, robotic, and/or the like. Further, aspects of this disclosure are described in terms of an embodiment using a medical system, such as the DA VINCI SURGICAL SYSTEM or ION SYSTEM commercialized by Intuitive Surgical, Inc. of Sunnyvale, California. Knowledgeable persons will understand, however, that aspects disclosed herein may be embodied and implemented in various ways, including robotic and, if applicable, non-robotic embodiments. Techniques described with reference to surgical instruments and surgical methods may be used in other contexts. Thus, the instruments, systems, and methods described herein may be used for humans, animals, portions of human or animal anatomy, industrial systems, general robotic, or teleoperational systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (with or without return to a human or animal anatomy) and for procedures on human or animal cadavers. Further, these techniques can also be used for medical treatment or diagnosis procedures that include, or do not include, surgical aspects.

Although illustrative embodiments have been shown and described, a wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the disclosure should be limited only by the following claims, and it is appropriate that the claims be construed broadly and, in a manner, consistent with the scope of the embodiments disclosed herein.

What is claimed is:

1. A system for adjusting an endoscope, the system comprising:
a memory; and
a controller communicatively coupled to the memory, wherein the controller:
moves the endoscope through a cannula;
stops the endoscope at a position in the cannula;
captures, using the endoscope at the position, images of a reference corresponding to the cannula; and
adjusts a parameter of the endoscope based on the reference in the images.

2. The system of claim 1, wherein stopping the endoscope comprises stopping a carriage on which the endoscope is positioned.

3. The system of claim 1, wherein the controller further prevents the endoscope from moving further through the cannula until after the endoscope captures the images.

4. The system of claim 1, wherein adjusting the parameter comprises:
dewarping the images to produce dewarped images;
comparing the dewarped images to determine a misalignment in the endoscope;
determining, based on a size of the reference and the misalignment in the endoscope, an adjustment; and
making the adjustment to the parameter.

5. The system of claim 4, wherein the parameter is an extrinsic parameter of the endoscope.

6. The system of claim 1, wherein the controller further:
determines at least one of a contrast or intensity of the reference in the images; and
adjusts a lumination of the endoscope based on at least one of the contrast or intensity.

7. The system of claim 1, wherein the reference comprises a marking on an inner wall of the cannula.

8. The system of claim 1, wherein the reference comprises an opening at an end of the cannula.

9. The system of claim 1, wherein stopping the endoscope at the position in the cannula is according to a marking on an inner wall of the cannula and at a predetermined distance away from the reference.

10. The system of claim 1, wherein the controller further:
determines a border of an image captured by the endoscope after the endoscope is positioned at a surgical site outside the cannula;
determines a difference between the border of the image and a reference border; and
generates, based on the difference exceeding a threshold, an alert indicating that the endoscope should be calibrated.

11. The system of claim 1, wherein the endoscope comprises a first camera and a second camera, wherein the images comprise a first image captured by the first camera and a second image captured by the second camera, and wherein adjusting the parameter comprises:
determining a pixel misalignment between the first image and the second image;
converting, based on a size of the reference, the pixel misalignment into a translational misalignment and a rotational misalignment; and
adjusting the parameter based on the translational misalignment and the rotational misalignment.

12. The system of claim 1, wherein the controller further measures a distance based on the adjusted parameter.

13. A method for adjusting an endoscope, the method comprising:
moving the endoscope through a cannula;
stopping the endoscope at a position in the cannula;
capturing, using the endoscope at the position, images of a reference corresponding to the cannula; and
adjusting a parameter of the endoscope based on the reference in the images.

14. The method of claim 13, wherein stopping the endoscope comprises stopping a carriage on which the endoscope is positioned.

15. The method of claim 13, further comprising preventing the endoscope from moving further through the cannula until after the endoscope captures the images.

16. The method of claim 13, wherein adjusting the parameter comprises:
dewarping the images to produce dewarped images;
comparing the dewarped images to determine a misalignment in the endoscope;
determining, based on a size of the reference and the misalignment in the endoscope, an adjustment; and
making the adjustment to the parameter.

17. The method of claim 16, wherein the parameter is an extrinsic parameter of the endoscope.

18. The method of claim 13, further comprising:
determining at least one of a contrast or intensity of the reference in the images; and
adjusting a lumination of the endoscope based on at least one of the contrast or intensity.

19. The method of claim 13, wherein the reference comprises a marking on an inner wall of the cannula.

20. A non-transitory machine-readable medium storing instructions for adjusting an endoscope that, when executed by a processor, cause the processor to:
move the endoscope through a cannula;
stop the endoscope at a position in the cannula;
capture, using the endoscope at the position, images of a reference corresponding to the cannula; and
adjust a parameter of the endoscope based on the reference in the images.
